(19) United States
(12) Patent Application Publication
(10) Pub. No.: US 2026/0215730 A1
(43) Pub. Date: Jul. 30, 2026

(54) DYNAMIC ALGOMETER

(71) Applicant: Knobzz UK LTD., Pinner (GB)

(72) Inventors: Simeon NIEL-ASHER, Raanana (IL); Yoram PONY, Moshav Ramat Raziel (IL)

(73) Assignee: Knobzz UK LTD., Pinner (GB)

(21) Appl. No.: 19/460,404

(22) Filed: Jan. 27, 2026

Related U.S. Application Data
(60) Provisional application No. 63/750,838, filed on Jan. 29, 2025.

(51) Int. Cl. A61B 5/00

(52) U.S. Cl. CPC A61B 5/4824; A61B 5/0053

(57) ABSTRACT

The presently disclosed subject matter relates to the field of pain management carried out by a dynamic durometer which seamlessly measures tissue hardness by means of a roller assembly head in contact with a patient. The invention provides systems and methods related to identifying trigger points, mapping the same, and implementing active pressure application to treat and alleviate symptoms

### FIELD OF THE INVENTION

The presently disclosed subject matter relates to the field of pain management carried out by a dynamic durometer which seamlessly measures tissue hardness by means of a roller assembly head in contact with a patient. The invention provides systems and methods related to identifying trigger points, mapping the same, and implementing active pressure application to treat and alleviate symptoms.

### BACKGROUND

Musculoskeletal pain and trigger points are commonly associated with areas of increased tissue hardness in muscles. Existing devices for assessing musculoskeletal pain are often static and limited in their ability to dynamically detect and measure tissue hardness or trigger points. The present invention provides a novel device and system for dynamic, real-time assessment of tissue hardness and musculoskeletal pain through a rolling mechanism integrated with a durometer.

The present device is an algometer which incorporates a durometer to measure tissue hardness and a roller assembly to facilitate the smooth motion of the durometer across the surface of a material. The device is used for diagnostics and therapy: pain relief, muscle pain reduction, etc.

### SUMMARY

In one embodiment the invention discloses a dynamic durometer device for pain management in an individual, the device comprising:
an indentor assembly comprising at least one displacement sensor, and configured to measure the indentation depth when pressed on the skin of the individual;
a roller assembly head attached at the distal end of the indentor assembly and configured to maintain contact with the skin whilst the indentor assembly measures the indentation depth; and
a chip comprising a controller configured to calculate tissue hardness based on the indentation depth.

In one embodiment the controller is further configured to:
calibrate the dynamic durometer device after the indentor assembly selectively applies pressure to the skin of the individual to provide a baseline tissue hardness value;
record tissue hardness values at different locations of the individual; and identify regions of elevated tissue hardness values in comparison with the baseline tissue hardness value.

In one embodiment the roller assembly head comprises at least one rollerball head, at least one axle-mounted cylindrical roller, or a combination thereof. In one embodiment the at least one displacement sensor is operatively coupled to each of the at least one rollerball head, the at least one axle-mounted cylindrical roller, or a combination thereof. In one embodiment the dynamic durometer further comprises a triangulation system comprising at least three displacement sensors and the controller, wherein the triangulation system is configured to identify regions of elevated tissue hardness values with greater resolution, enhance the accuracy and precision of the indentation depth measurement, or a combination thereof. In one embodiment the roller assembly head is coupled to a gimbal system or a ball-and-socket system, and thereby configured to rotate about multiple axes to enable multidirectional movement. In one embodiment the dynamic durometer further comprises a display unit configured to display the depth of indentation, tissue hardness value, or a combination thereof. In one embodiment the at least one rollerball head has a diameter ranging between 0.5 cm to 5 cm. In one embodiment the dynamic durometer further comprises a housing structure comprising the indentor assembly within it and wherein the roller assembly head protrudes from the housing structure. In one embodiment the dynamic durometer further comprises a handle attached to the housing structure.

In one embodiment the dynamic durometer further comprises an ultrasound unit operatively coupled to the roller assembly head and configured to deliver ultrasound, vibrations, or a combination thereof, to the individual when the roller assembly head is in contact with the individual. In one embodiment the roller assembly head further comprises a light source configured to deliver light into the skin of the individual when the roller assembly head is in contact with the individual. In one embodiment the light source is a laser.

In one embodiment the dynamic durometer further comprises a temperature regulation system operatively coupled to the roller assembly head and configured to maintain a predetermined temperature for the roller assembly head. In one embodiment the dynamic durometer further comprises a gyrometer configured to monitor the orientation, spatial rotation, spatial location, and angular velocity of the dynamic durometer. In one embodiment the dynamic durometer further comprises an indicator element operatively coupled to the controller and configured to provide haptic, visual, or auditory feedback to a user upon detection of a predetermined tissue hardness. In one embodiment the controller is further configured to direct the indentor assembly to selectively apply a predetermined pressure at or near one or more locations with elevated tissue hardness, thereby reducing pain in the individual. In one embodiment the dynamic durometer further comprises a power source.

In one embodiment the chip further comprises:
an analog-to digital converter (ADC) configured to convert tissue hardness data into digital data;
an encoder configured to encode the digital data, resulting in encoded digital data;
a memory unit configured to store the encoded digital data; and
a communication module comprising a wireless transmitter operatively coupled to the controller and configured to send and receive the encoded digital data to an external system;
or a combination thereof.

In one embodiment the invention provides a pain management system comprising:
the dynamic durometer of the invention; and
an external system configured to send and receive the encoded digital data.

In one embodiment the wireless transmitter is compatible with Bluetooth and/or WiFi communication. In one embodiment the memory unit is a physical memory storage unit, cloud storage, or a combination thereof.

In one embodiment the external system comprises:
a data receiver configured to receive the encoded digital data transmitted from the dynamic durometer;
a processor configured to:
process tissue hardness data received from the wireless transmitter;
execute software applications based on the encoded digital data received from the wireless transmitter whereby diagnostic metrics and therapeutic metrics are determined;
remotely control or adjust settings of the dynamic durometer system based on user preferences or predetermined parameters;
and a memory configured to store the received data.

In one embodiment the chip is further configured to onboard any of the following data related to the individual selected from: age, sex, fitness level, height, weight, medical history, current medications, allergies, dietary habits, exercise routine, smoking routine, alcohol consumption, sleep patterns, chronicity of condition, pain location, pain onset, pain duration, physical limitations, fatigue levels, symptom triggers, heart rate, blood pressure, glucose level, mobility assessment, and injury history; and
wherein the controller is further configured to:
analyze the onboarded data to categorize the individual into a predefined target group; and
generate a personalized pain management scheme to direct the dynamic durometer to selectively apply a predetermined pressure at or near one or more locations with elevated tissue hardness, thereby reducing pain in the individual.

In one embodiment the personalized pain management scheme defines the pressure, location, and duration of the application of the dynamic durometer, which is thereby implemented by the dynamic durometer.

In one embodiment the chip is further configured to carry out any of the following selected from:
analyze tissue hardness data to increase the spatial resolution of locations with elevated tissue hardness;
apply a data compression algorithm to reduce the size of data for storage and transmission;
encode tissue hardness data with a timestamp.

In one embodiment the invention provides a method of pain management by administering pressure treatment to an individual, the method comprising:
providing the dynamic durometer system of claim 20;
detecting pressure applied to the roller assembly head at different locations within a treatment area on the individual;
identify regions of the skin with elevated tissue hardness values; and
applying a predetermined pressure profile by the roller assembly head to the individual at or near one or more locations with elevated tissue hardness, thereby administering the pressure treatment.

In one embodiment the predetermined pressure profile comprises:
a gradual pressure increase to a maximum predetermined threshold;
a constant pressure for a predetermined duration;
a gradual pressure decrease to a minimum predetermined threshold; or
a combination thereof.

In one embodiment the method further comprises performing a pre-scan procedure prior to detecting pressure applied to the roller assembly head. In one embodiment the pre-scan comprises determining a baseline pressure for the treatment area. In one embodiment the pre-scan comprises constructing a pressure map of the treatment area. In one embodiment the method further comprises orienting the controller on the treatment area based on the pressure map. In one embodiment the pre-scan comprises scanning a sample of the treatment area to determine a predicted baseline pressure map for the treatment area. In one embodiment the method further comprises rotating the roller assembly head about an axis. In one embodiment the pressure profile comprises a variable pressure for a predetermined amount of time. In one embodiment the method further comprises applying photobiomodulation, ultrasound treatment, vibration, temperature treatment, transcutaneous electrical nerve stimulation, electrical muscle stimulation, and/or shockwave therapy to the individual. In one embodiment the method further comprises transmitting usage information to the external system. In one embodiment the method further comprises receiving information comprising treatment recommendations from an outside system.

In one embodiment the remote device is further configured to display the tissue hardness values as a 3D body map on the remote device. In one embodiment the controller is further configured to provide real-time feedback in the form of haptic, audio, visual, or a combination thereof, to indicate the arrival at a trigger point.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1A is a photograph of a durometer with a distal end that contacts the skin of an individual. FIG. 1B is a side view of the durometer in FIG. 1A.

FIG. 1C is a schematic illustration of a rollerball head that attaches to the distal end of the durometer. FIG. 1D is a cylindrical roller head which attaches to the distal end of the durometer. FIG. 1E is a schematic of one version of the indentor assembly, together with the roller assembly.

FIG. 2 is a perspective view of an example of a roller assembly head of a massage device according to the presently disclosed subject matter.

FIG. 3 illustrates another example of a roller assembly head of the massage device according to the presently disclosed subject matter.

FIG. 4 illustrates a massage system according to the presently disclosed subject matter;

FIG. 5 and FIG. 6 are examples of data presented on the computing device of the system illustrated in FIG. 4.

FIGS. 7A through 8 illustrate an area of pain experienced by a patient and a corresponding trigger point.

FIG. 9 illustrates a sleeve comprising the massage device according to the presently disclosed subject matter.

FIG. 10 illustrates a facemask comprising the massage device according to the presently disclosed subject matter.

FIG. 11 illustrates a glove comprising the massage device according to the presently disclosed subject matter.

FIG. 12 illustrates an apparatus configured to be secured to a chair and comprising the massage device according to the presently disclosed subject matter.

FIG. 13 illustrates a shoulder compression wrap comprising the massage device according to the presently disclosed subject matter.

FIGS. 14A and 14B illustrate a headrest and a neck pillow comprising the massage device according to the presently disclosed subject matter.

FIGS. 15A and 15B illustrate a wrist rest comprising the massage device according to the presently disclosed subject matter.

FIG. 16 illustrates a mat comprising the massage device according to the presently disclosed subject matter.

FIG. 17 illustrates a ball comprising the massage device according to the presently disclosed subject matter.

FIG. 18 illustrates examples of weighted objects comprising the massage device according to the presently disclosed subject matter.

FIG. 19 illustrates a boot comprising the massage device according to the presently disclosed subject matter.

FIG. 20 shows a treatment algorithm flowchart.

FIG. 21 shows one example of a personalized treatment workflow.

FIG. 22 shows components of the dynamic durometer system.

FIG. 23 shows the components within a tracking system.

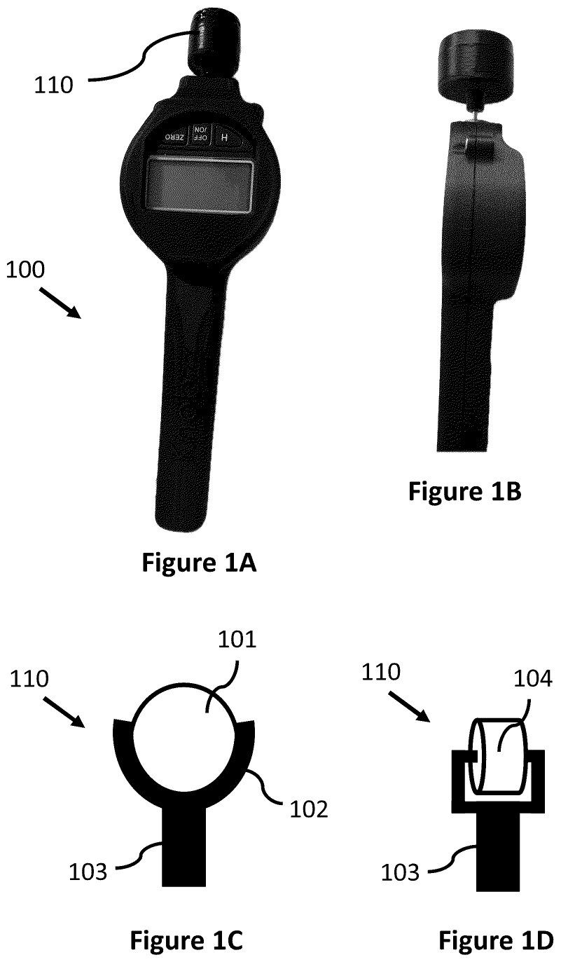

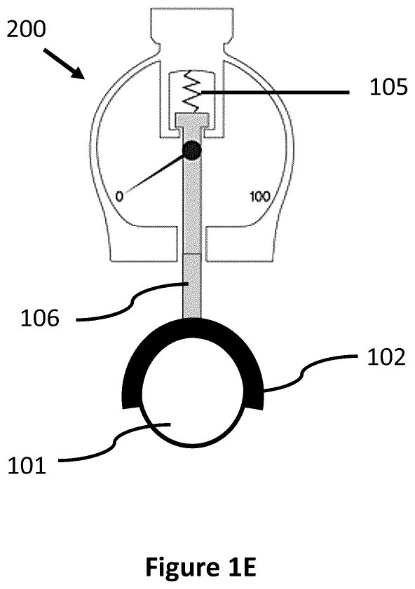

For simplicity and clarity of illustration, elements shown in the figures are not necessarily drawn to scale, and the dimensions of some elements may be exaggerated relative to other elements. In addition, reference numerals may be repeated among the figures to indicate corresponding or analogous elements.

### DETAILED DESCRIPTION

The present invention relates to a dynamic algometer that incorporates a roller assembly (e.g., a roller ball head) attachment and a durometer to assess tissue hardness, musculoskeletal pain, and locate trigger points. The rollerball, which may be of varying sizes, is mounted on a durometer to enable rolling over the skin. Areas of maximal tissue hardness correlate to areas of maximal pain. The device is equipped with force-detection capabilities and may include Bluetooth or Wi-Fi connectivity to transmit data to a digital platform. Additionally, it can feature input and output connections to solenoids or motors for active force application and feedback. The terms “algometer” and “durometer” are used interchangeably herein since the tissue hardness measured by the durometer directly relates to the corresponding pain that an individual experiences. Points of elevated tissue hardness are generally referred to herein as “trigger points”. Trigger points are specific areas of tightness or hyperirritability in muscles and connective tissues that are often associated with pain and dysfunction. They are sometimes described as “knots” within the muscle and are characterized by localized areas of hardened or tense muscle fibers.

There are different types of trigger points such as:
active trigger points which cause pain even without stimulation and are often linked to chronic pain conditions;
latent trigger points which do not cause pain unless pressure is applied thereon.

Trigger points can cause restricted muscle movement, lead to muscle weakness, cause stiffness, decreased flexibility, impaired circulation, etc. The invention described herein is directed to the use of a durometer to identify trigger points as well as utilizing pressure actuated by the device itself to relieve pain. Such therapy is analogous with massage therapy and/or trigger point release therapy. The methods and systems of the present invention are thus directed at any of the following: pain relief, pain management, muscle relaxation, reduction of muscle tension, improvement of blood circulation, relief from muscle stiffness, enhanced range of motion, stress relief, decrease in inflammation, improved bodily fluid drainage, etc.

The dynamic durometer of the present invention is typically used for measuring tissue hardness although it can be analogously used to measure the hardness of any material. Standard durometers tend to measure the hardness of materials such as elastomers, polymers, and rubbers. Durometers function by pressing an indentor into the material, measuring the depth of indentation under a specific force and calculating the hardness. Hardness is typically measured in the Shore scale. For example, Shore A, Shore D, Shore O, Shore OO, and Shore E—each corresponding to a different hardness range. The dynamic durometer of the present invention can measure hardness in terms of a Shore scale, any relative force scale, or absolute hardness, etc.

A primary goal of the present invention is to use a roller assembly to move the durometer across the surface of a material (e.g., skin of an individual) to ensure that contact is smoothly maintained throughout the pressing of the durometer. A practitioner (or the individual him/herself) can press the dynamic durometer, moving it across the skin. When there are changes to the tissue hardness under the skin, the individual will feel variations in pain, and can identify these points with the practitioner for further investigation. The indentor assembly is configured to calculate the tissue hardness and monitor where trigger points, or points of elevated tissue hardness, are located. The practitioner can then focus in on those trigger points, with a specific pressure profile program being implemented to actively target the trigger points by carrying out predetermined pressing on or near the trigger points themselves, or at corresponding points associated with the trigger points, thereby inducing pain relief.

It has been found that target locations, for example trigger points associated with pain experienced by the patient, may be characterized by an elevated pressure. Accordingly, the dynamic durometer device is configured to identify target locations by scanning the treatment area for locations of elevated pressure which indicate the presence of a target location. Identification of a target location is performed using the same element, e.g., a roller assembly head or other element in contact with or directly bearing on the patient, which applies the pressure.

It will be appreciated that a detected pressure may be considered “elevated” to associate it with a target location if it exceeds one or more reference pressures. A reference pressure may be determined in any suitable value. According to some examples, a reference pressure is determined based on one or more pressures measured at other locations in the treatment area. According to some examples, a reference pressure is determined based on a predetermined expected value for the pressure at the location, e.g., based on the contours of the treatment area, for example as described above.

According to some examples, a detected pressure is considered “elevated” if it exceeds the one or more reference pressures by a predetermined amount.

It will be further appreciated that minor elevations in pressure may be expected, and large elevations in pressure may be due to something other than a trigger point (e.g., the pressure detection arrangement may detect a pressure which is elevated by relatively large amount when the roller assembly head exerts pressure on protruding hard tissue, such as a shoulder blade). Accordingly, the controller may be configured to identify a target location where the pressure detection arrangement detects a pressure which is elevated by an amount which falls within a predetermined range. According to some examples, the range may be closed, i.e., a detected pressure must be elevated by an amount which exceeds a minimum value but does not exceed a maximum value in order to indicate a target location. According to other examples, the range is open-ended, i.e., it defines either a minimum amount or a maximum amount by which a detected pressure must be elevated in order to indicate a target location. The minimum and/or maximum values of the range may differ based on the location of the treatment area (e.g., based on its anatomy, based on historical data, etc.), based on information about the patient (e.g., weight, height, gender, user history, etc.), or on any other suitable factors.

The present invention provides a dynamic durometer for pain management in an individual. As understood herein “dynamic durometer” refers to a durometer that measures hardness whilst moving, in contact, along the surface, typically of the skin of an individual. An expert will understand that such a dynamic durometer can be used for measuring hardness of any material. The “dynamic” nature of the durometer refers to its ability to move whilst making measurements, the movement being enabled by a roller assembly.

In one embodiment the invention provides a dynamic durometer for pain management in an individual, the device comprising:
an indentor assembly comprising at least one displacement sensor, and configured to measure the indentation depth when pressed on the skin of the individual;
a roller assembly head attached at the distal end of the indentor assembly and configured to maintain contact with the skin whilst the indentor assembly measures the indentation depth; and
a chip comprising a controller configured to calculate tissue hardness based on the indentation depth.

In one embodiment the invention provides a dynamic durometer for measuring hardness in a material, the device comprising:
an indentor assembly comprising at least one displacement sensor, and configured to measure the indentation depth when pressed on the material;
a roller assembly head attached at the distal end of the indentor assembly and configured to maintain contact with the material whilst the indentor assembly measures the indentation depth; and
a chip comprising a controller configured to calculate hardness of the material based on the indentation depth.

As understood herein “pain management” refers to any type of improvement to an individual's experience of pain such as, but not limited to: pain relief, muscle relaxation, reduction of muscle tension, improvement of blood circulation, relief from muscle stiffness, enhanced range of motion, stress relief, decrease in inflammation, improved bodily fluid drainage. Thus, pain management includes providing physical therapy to an individual, whether the individual is affected by a particular condition or not. In other embodiments the dynamic durometer is for use in an individual affected by musculoskeletal conditions, neurological conditions, circulatory conditions, lymphatic conditions, sports-related injuries, chronic conditions, post-surgical rehabilitation, stress-related conditions, etc. Examples of musculoskeletal conditions include, but are not limited to: back pain, neck pain, stiffness, shoulder pain, rotator cuff injuries, frozen shoulder, knee pain, hip pain or stiffness, muscle strain, muscle sprains, tendinitis, postural imbalance, and trigger points. Examples of neurological conditions include, but are not limited to: nerve compression, neuropathies, spasticity, muscle hypertonic, and peripheral nerve injury. Examples of circulatory conditions include, but are not limited to: poor circulation, chronic venous insufficiency, high blood pressure, low blood pressure, swelling, and blood clots. Examples of chronic conditions include, but are not limited to: arthritis, osteoarthritis, rheumatoid arthritis, fibromyalgia, and chronic pain syndrome. Other examples of the use of the dynamic durometer include: tension headaches, migraines, jaw pain, stress-induced muscle tension, and whiplash.

The “indentor assembly” refers to any indentation means configured to facilitate a force measurement when pressed on a surface. Such means include a spring-loaded mechanism. In some embodiments the indentor assembly comprises any of the following selected from: a spring-loaded mechanism, an indentor tip (also referred to as a ‘head’), a guide sleeve configured to align and constrain the motion of the indentor head along a predefine axis, a force application system operatively connected to the spring-loaded mechanism to transmit the applied force to the indentor head, and at least one displacement sensor.

As understood herein the “chip” refers to a physical electronic component configured to process data and execute instructions. A chip may include any of the following components:
a micro/controller unit configured to, at least, perform calculations related to indentation depth and correlate the data with the tissue hardness value;
a memory unit configured to, at least, comprise volatile and/or non-volatile memory, to store operational instructions, calibration data, and hardness calculation algorithms, etc.;
an input/output interface, at least, configured to receive signals from sensors, or other peripheral devices, and transmit processed data to a display or external system;
a power management module, at least, configured to regulate and distribute power within the chip, and the dynamic durometer, for efficient operation; and
a signal processing unit, at least, configured to filter, amplify, and digitize sensor input signals for subsequent analysis by the controller (or other component e.g., remote system);
or a combination thereof.

The displacement sensor is configured to measure the indentation depth of the roller assembly head. Thus, typically, the indentor assembly measures the indentation depth by means of the displacement sensor, and the controller calculates the tissue hardness based on the indentation depth.

In one embodiment the controller is further configured to carry out any of the following selected from:
calibrate the dynamic durometer device after the indentor assembly selectively applies pressure to the skin of the individual to provide a baseline tissue hardness value;
record tissue hardness values at different locations of the individual; and
identify regions of elevated tissue hardness values in comparison with the baseline tissue hardness value.

The dynamic durometer device selectively applies pressure to the skin of the individual by utilizing a controlled indenter assembly that can adjust the amount of force exerted based on predefined parameters. In various embodiments this is achieved by a motorized and/or spring-loaded mechanism and a feedback system that monitors the applied pressure in real-time. The device ensures consistent and precise force application by incrementally increasing or modulating the pressure until the desired force threshold is reached. This selective application of pressure allows the dynamic durometer to adapt to variations in skin elasticity, underlying tissue characteristics, or environmental factors, ensuring accurate and reproducible measurements. The process is calibrated to provide a baseline tissue hardness value, which serves as a reference for subsequent assessments or comparisons.

The recording of tissue hardness values can be done via an internal storage, or a cloud based storage. An archive of such data can be stored, for future analysis and pressure profile implementation optimization. Thus, libraries of data can be accumulated and stored with proprietary data related to any or all dynamic durometer measurements.

In one embodiment the roller assembly head comprises at least one rollerball head, at least one axle-mounted cylindrical roller, or a combination thereof. A rollerball head is configured to rotate freely and provide contact with the skin of the individual. Typically, it is located in a retaining house configured to encase the rollerball head. As such, and in various embodiments, the retaining house comprises an internal bearing mechanism to reduce friction and ensure smooth rotation and motion.

In one embodiment the at least one displacement sensor is operatively coupled to each of the at least one rollerball head, the at least one axle-mounted cylindrical roller, or a combination thereof. Any number of rollerball heads and/or axle-mounted cylindrical rollers can be used alone or in combination.

In one embodiment the dynamic durometer further comprises a triangulation system comprising at least three displacement sensors and the controller, wherein the triangulation system is configured to identify regions of elevated tissue hardness values with greater resolution, enhance the accuracy and precision of the indentation depth measurement, or a combination thereof. Utilizing a combination or displacement sensors mounted individually on roller assembly heads enables the triangulation of the tissue hardness signal. It can provide higher resolution of measurements, increased accuracy, and precision.

In one embodiment the roller assembly head is coupled to a gimbal system or a ball-and-socket system, and thereby configured to rotate about multiple axes to enable multidirectional movement. In one embodiment the dynamic durometer further comprises a display unit configured to display the depth of indentation, tissue hardness value, or a combination thereof. The display unit can be further configured to display any data relevant to the dynamic durometer device, the measurements it makes, or user data relevant for pain management. The display unit can be further configured to interact with the user or practitioner. For example: real-time feedback, user prompts and instructions, customization options, alerts and notifications, interactive touch screen controls, data storage and retrieval, visual/audio/tactile cues, diagnostic information, connectivity features, user-guided support, etc.

In one embodiment the at least one rollerball head has a diameter ranging between 0.5 cm to 5 cm. In one embodiment the at least one rollerball head has a diameter ranging between 0.5 cm to 2 cm.

In one embodiment the dynamic durometer further comprises a housing structure comprising the indentor assembly within it and wherein the roller assembly head protrudes from the housing structure. In one embodiment the dynamic durometer further comprises a handle attached to the housing structure. The handle can be ergonomically configured to improve the useability of the device.

In one embodiment the dynamic durometer further comprises an ultrasound unit operatively coupled to the roller assembly head and configured to deliver ultrasound, vibrations, or a combination thereof, to the individual when the roller assembly head is in contact with the individual.

In one embodiment the roller assembly head further comprises a light source configured to deliver light into the skin of the individual when the roller assembly head is in contact with the individual. Examples of light sources include: laser, and LED.

In one embodiment the durometer further comprises a temperature regulation system operatively coupled to the roller assembly head and configured to maintain a predetermined temperature for the roller assembly head. In turn the roller assembly unit, being in contact with the of the individual, increases or decreases the temperature of the skin depending on the temperature setting of the temperature regulation system. Thus, heat therapy or cold therapy can be locally delivered to targeted areas of the skin.

In one embodiment the dynamic durometer further comprises a gyrometer configured to monitor the orientation, spatial rotation, spatial location, and angular velocity of the dynamic durometer.

In one embodiment the dynamic durometer further comprises an indicator element operatively coupled to the controller and configured to provide tactile, visual, or auditory feedback to a user upon detection of a predetermined tissue hardness. In one embodiment the indicator element is comprised within the display unit. Tactile (e.g., vibrations or pulses), visual (e.g., flashing lights, color changes, or dynamic graphical indicators), or auditory cues (e.g., beeping, tones, or voice prompts) can be employed to convey changes in the device's state, operational status, or user-relevant information. These cues can provide feedback such as successful measurement completion, warnings for improper usage, system readiness, changes in applied pressure, or alerts for calibration requirements, etc.

In one embodiment the controller is further configured to direct the indentor assembly to selectively apply a predetermined pressure at or near one or more locations with elevated tissue hardness, thereby reducing pain in the individual. In some embodiments, the application of a predetermined pressure is not necessarily carried out at or near the locations with elevated tissue hardness (e.g., trigger points), but rather at any location that causes the reduction of pain at the trigger points. Trigger points are hypersensitive spots in muscles or fascia that can cause localized or referred pain. Some trigger points, known as satellite trigger points, develop in muscles affected by pain or tension from a primary trigger point. Treating these satellite points can sometimes relieve pain in the primary area. Trigger points often have referral patterns, where pain is felt in a different location than the trigger point itself. This occurs due to neural pathways, myofascial connections, or reflex responses, where stimulating certain areas, like the palms, can influence tension or pain in distant regions, such as the neck. As such, the application of predetermined pressure can be carried out at any of these locations, for pain management.

In one embodiment the dynamic durometer further comprises a power source. Examples of the power source include, but are not limited to: a battery, or an external power source connected to the mains.

In one embodiment the chip further comprises:
an analog-to digital converter (ADC) configured to convert tissue hardness data into digital data;
an encoder configured to encode the digital data, resulting in encoded digital data;
a memory unit configured to store the encoded digital data; and
a communication module comprising a wireless transmitter operatively coupled to the controller and configured to send and receive the encoded digital data to an external system;
or a combination thereof.

The ability to collect and analyze large volumes of data over time allows the user to identify trends in both diagnostics and treatments. The present invention leverages this stored data and performs longitudinal analysis to create comprehensive, personalized records of a patient's progress. By identifying trends and correlating muscle function with pain responses, therapists and users can design more effective, targeted treatment plans while continuously optimizing pain management strategies. Consequently, the present invention integrates real-time diagnostics with analytics to deliver optimized pain management solutions. The data accumulated can be stored in a cloud-based archive and analyzed using machine learning algorithms to predict future outcomes and refine treatment approaches.

In one embodiment the invention provides a pain management system comprising:
the dynamic durometer of the present invention; and
an external system configured to send and receive the encoded digital data.

In one embodiment the wireless transmitter is compatible with Bluetooth and/or WiFi communication. In one embodiment the memory unit is a physical memory storage unit, cloud storage, or a combination thereof.

In one embodiment the external system comprises:
a data receiver configured to receive the encoded digital data transmitted from the dynamic durometer;
a processor configured to:
   process tissue hardness data received from the wireless transmitter;
   execute software applications based on the encoded digital data received from the wireless transmitter whereby diagnostic metrics and therapeutic metrics are determined;
   remotely control or adjust settings of the dynamic durometer system based on user preferences or predetermined parameters;
and a memory configured to store the received data.

In one embodiment chip is further configured to onboard any of the following data related to the individual selected from: age, sex, fitness level, height, weight, medical history, current medications, allergies, dietary habits, exercise routine, smoking routine, alcohol consumption, sleep patterns, chronicity of condition, pain location, pain onset, pain duration, physical limitations, fatigue levels, symptom triggers, heart rate, blood pressure, glucose level, mobility assessment, and injury history; and
wherein the controller is further configured to:
analyze the onboarded data to categorize the individual into a predefined target group; and
generate a personalized pain management scheme to direct the dynamic durometer to selectively apply a predetermined pressure at or near one or more locations with elevated tissue hardness, thereby reducing pain in the individual.

Predefined target groups can include any of the following selected from: age-group, fitness level, pain severity, condition-specific group, health risk group, lifestyle-based group, sleep-related group, injury history group, medical history group, etc. Categorizing individuals into groups provides a helpful baseline for personalized pain management, which can then be optimized and updated in real time throughout treatment. See examples.

As used herein the “personalized pain management scheme” refers to a tailored regimen to treating, alleviating symptoms in, or reducing pain for, any of the indicators of pain to meet the pain management needs of the individual.

In one embodiment the personalized pain management scheme defines the pressure, location, and duration of the application of the dynamic durometer, which is thereby implemented by the dynamic durometer. The pain management scheme can be comprised within any number of sessions. For example, the individual may require 10 sessions of dynamic durometer treatment. Each session can be tailored differently according to the changes in pain status of the individual, and the requirements of the treatment.

In one embodiment the chip is further configured to carry out any of the following selected from:
analyze tissue hardness data to increase the spatial resolution of locations with elevated tissue hardness;
apply a data compression algorithm to reduce the size of data for storage and transmission;
encode tissue hardness data with a timestamp.

In one embodiment the invention provides a method of pain management by administering pressure treatment to an individual, the method comprising:
providing the dynamic durometer system of the invention;
detecting pressure applied to the roller assembly head at different locations within a treatment area on the individual;
identify regions of the skin with elevated tissue hardness values; and
applying a predetermined pressure profile by the roller assembly head to the individual at or near one or more locations with elevated tissue hardness, thereby administering the pressure treatment.

In one embodiment the predetermined pressure profile comprises:
a gradual pressure increase to a maximum predetermined threshold;
a constant pressure for a predetermined duration;
a gradual pressure decrease to a minimum predetermined threshold; or
a combination thereof.

As understood herein “pressure profile” refers to a pattern or sequence of pressure application during a treatment process. In one embodiment predetermined pressure profile comprise three stages: pressure increase, constant pressure, and pressure decrease. The predetermined pressure profile may be repeated on the same spot any number of times. In one embodiment the predetermined pressure profile further comprises any of the following selected from: pulsing, intermittent pressure, and waveform pressure. Examples of waveform pressure include, but are not limited to: sinusoidal, square, triangular, and pulse. Depending on the predefined target group, each pressure profile can be analyzed to determine the optimized pressure profile. Iterative processing can be used to optimize the pain management scheme.

In one embodiment the predetermined pressure profile is carried out a plurality of times. In one embodiment the predetermined pressure profile is carried out at least once. In one embodiment the predetermined pressure profile is carried out between 1 to 10 times. In one embodiment the predetermined pressure profile is carried out between 1 to 100 times. In one embodiment the predetermined pressure profile is carried out between 1 to 1000 times. In one embodiment the pain management scheme is comprised within any number of treatment sessions. In one embodiment the method further comprises massage.

In one embodiment the method further comprises performing a pre-scan procedure prior to detecting pressure applied to the roller assembly head. In one embodiment the pre-scan comprises determining a baseline pressure for the treatment area. In one embodiment the pre-scan comprises constructing a pressure map of the treatment area. In one embodiment the method further comprises orienting the controller on the treatment area based on the pressure map. In one embodiment the pre-scan comprises scanning a sample of the treatment area to determine a predicted baseline pressure map for the treatment area. In one embodiment the method further comprises rotating the roller assembly head about an axis. In one embodiment the pressure profile comprises a variable pressure for a predetermined amount of time. In one embodiment the method further comprises applying photobiomodulation, ultrasound treatment, vibration, temperature treatment, transcutaneous electrical nerve stimulation, electrical muscle stimulation, and/or shockwave therapy to the individual.

In one embodiment the method further comprises transmitting usage information to the external system. In one embodiment the method further comprises receiving information comprising treatment recommendations from an outside system. In one embodiment the remote device is further configured to display the tissue hardness values as a 3D body map on the remote device. In one embodiment the controller is further configured to provide real-time feedback in the form of haptic, audio, visual, or a combination thereof, to indicate the arrival at a trigger point.

The figures will now be described. It should be understood that the terms “massage head” and “roller assembly head” can be understood analogously. Although the dynamic durometer is primarily used for pain management, for example, it can be understood that the roller assembly head can function, where needed, as a massage head for the purposes of massaging. In view of this, the terms can be understood interchangeably.

FIG. 2 shows a massage head which comprises two or more projections 20, each for bearing on the patient. According to examples in which the massage head 16 comprises two more or projections 20, the massage head may be configured to apply pressure to more than one location simultaneously. It will be appreciated that while the massage head 16 illustrated in FIG. 2 comprises two projections 20, the massage device 10 may comprise a massage head having any suitable number of projections, of similar or varying size, shape, etc.

The massage head 16 and projections 20 may be provided according to any suitable design. According to some examples, for example as illustrated in FIG. 3, the massage head 16 and/or projection 20 may comprise a ball bearing, e.g., configured as a pot or transfer bearing, for bearing on the patient.

The massage head 16 may be configured to rotate about an axis. According to some examples, the massage head 16 comprises a suitable mechanism for the rotation. According to other examples, the driving arrangement 18 is configured to rotate the massage head 16. According to examples in which the massage head 16 comprises two more or projections 20, the rotation may be about an axis located between the projections.

According to examples in which the massage head comprises two or more projections 20, the massage head may comprise an actuation arrangement (not illustrated), configured to move one or more of the projections on the massage head 16. According to some examples, the actuation arrangement is configured to change the lateral distance between two or more of the projections 20.

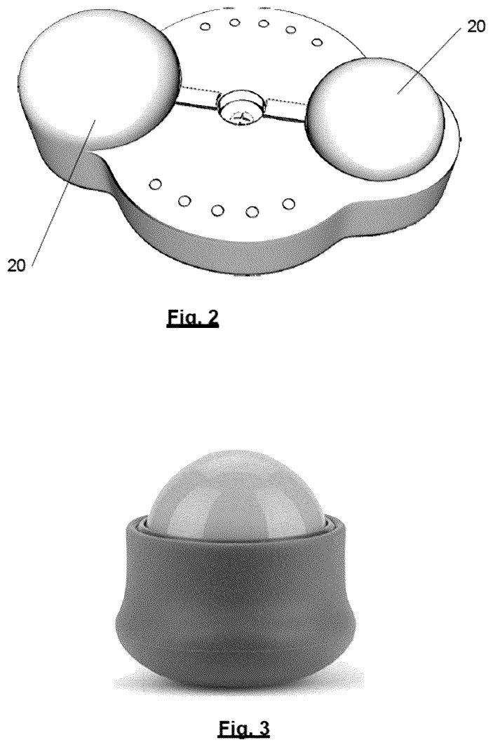

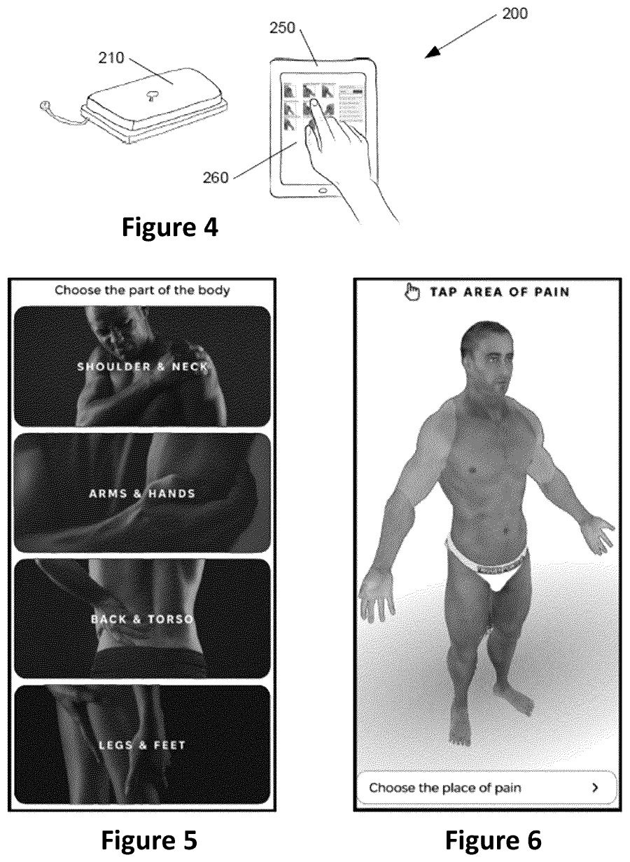

As illustrated in FIG. 4, a massage system, which is generally indicated at 200, may be provided. According to some examples, the massage system may be configured to facilitate a user (i.e., a patient) to administer a treatment to himself, e.g., without direct involvement of a professional. The massage system 200 may comprise a massage device 210 similar to that described above with reference to and as illustrated in FIGS. 2-3, and a computing device 250. The computing device 250 may be a dedicated device, or it may be implemented as software installed on a general-purpose computer, for example as an application running on a smartphone, tablet, etc.

The computing device 250 may comprise a user interface 260, on which it prompts the patient to indicate the area where they are experiencing pain. The user interface may be a touchscreen, thereby receiving input and displaying output on a single interface. As illustrated in FIGS. 5 and 6, the computing device 250 may be configured to present several general anatomical areas (e.g., back of arm, front of shoulder, mid-thoracic back, etc.) and/or specific areas (e.g., deltoid, levator scapulae). These areas may be accompanied by figures illustrating the areas to aid in patient selection. Once the area in which pain is experienced is indicated, the computing device 250 determines a corresponding trigger point to which a treatment may be applied to address the pain in the indicated area. A database of anatomical areas and corresponding trigger points may be stored in a memory of the computing device 250 and/or be accessed by the computing device from a remote computing device or storage system.

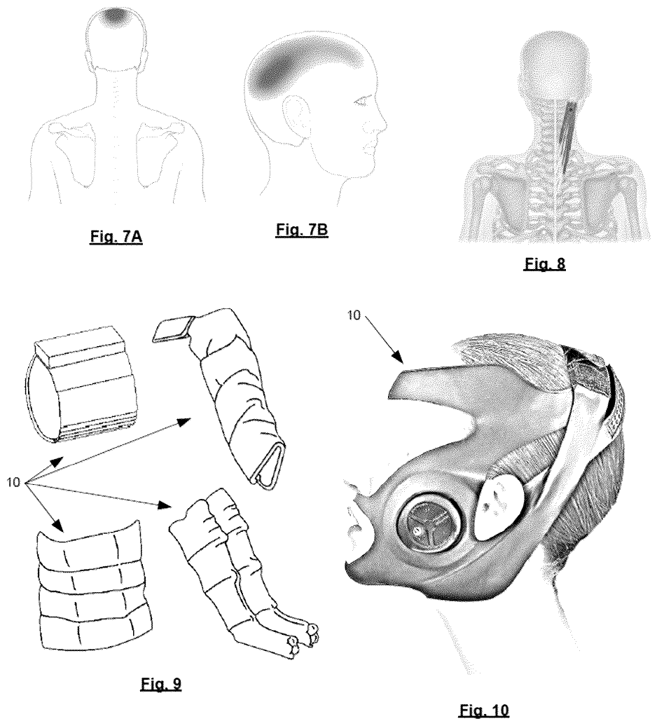

It will be appreciated that the trigger point is not necessarily in the same area of the body in which pain is experienced. For example, pain experienced in the vertex of the skull, e.g., as illustrated in FIGS. 7A and 7B may correspond with a trigger point on the splenius capitis muscle in the neck, as illustrated in FIG. 8. While the trigger point in general corresponds to a target location within the treatment area, it is only approximate, the specific location of the target location identified by the massage device 210, for example as described above, may be in a slightly different than indicated, based on a number of anatomical and other factors (e.g., posture, weight bearing, etc.).

The computing device 250 may then display instructions, e.g., via the user interface, on which part of the body the massage device 210 should be placed. The massage device 210 is operated to scan the treatment area and apply a treatment, for example as described above.

According to some examples, the controller may be configured to communicate with an external system, e.g., over the internet, a telephony connection, private network, etc., to transmit information thereto and to receive information therefrom. The information transmitted may include, but is not limited to, data relating to identified target locations, pressure maps, treatments applied, user feedback (e.g., pain rating before and/or after treatment), etc. The information received may be personalized to the patient, thereby facilitating personalized treatment based, e.g., on provided parameters of the patient, including, but not limited to, age, weight, height, sex, pre-existing conditions, medical history, information about previous uses of the device, recommendations, etc.

According to some examples, the external system may analyze data from many users, including feedback, and make one or more recommendations for treatment based on parameters of a patient. According to some examples, a machine leaning model may be trained using the data from many users to provide the recommendations. The recommendations may include, but are not limited to, parameters of the pressure profile for example as described above, rotation speed, lateral distance between projections 20 of the massage head, expected values for pressure detection, use of auxiliary treatment, etc.

The massage device 10 as described above, including, but not limited to, when constituting part of the system 200 described above with reference to and as illustrated in FIG. 4, may be implemented in any suitable manner. According to some examples, it constitutes part of a substantially planar device which may be secured to the patient before use. According to some examples, it constitutes part of a sleeve, examples of which are illustrated in FIG. 9, which may be secured around a limb and/or a torso of the patient. The massage device 10 may be comprise pockets for receipt therein of a cooling or heating material, for example for compression therapy. A massage device 10 according to these examples may be used, e.g., to treat lateral epicondylalgia, shoulder/upper arm pain, hamstring pain, calf pain, torso pain, etc.

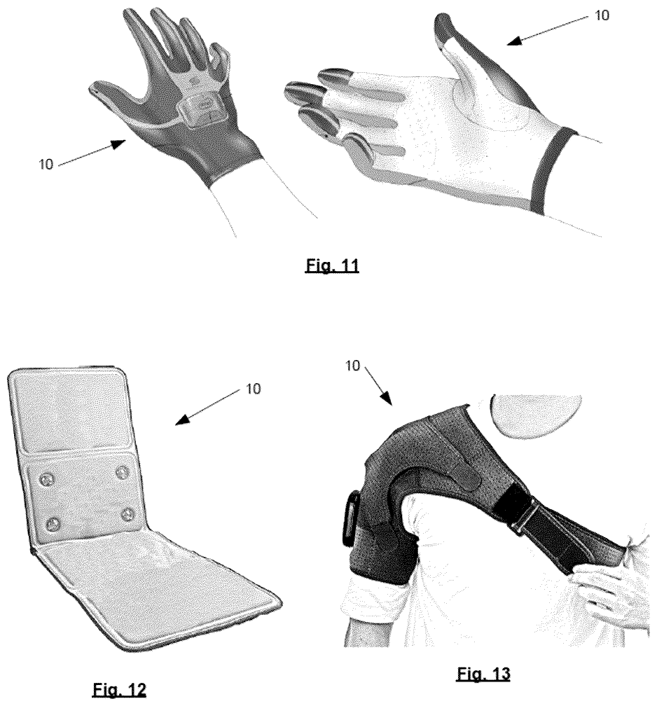

According to some examples, for example as illustrated in FIG. 10, the massage device 10 may be provided as part of a facemask, with functional elements (massage assembly, pressure detection arrangement, etc.) contained therewithin. The massage device 10 according to these examples may be configured, e.g., to apply treatment to the masseter and/or temproalis muscles. According to some examples, it may be used to treat temporomandibular joint disorder.

According to some examples, for example as illustrated in FIG. 11, the massage device 10 may be integrated into a glove, with functional elements (massage assembly, pressure detection arrangement, etc.) contained therewithin. According to some examples, a single pressure detection arrangement is provided. According to some examples, several pressure detection arrangements are provided, e.g., each associated with a different finger of the glove to measure the pressure applied thereto. Each of the several pressure detection arrangements may be configured to measure the pressure applied to a finger independently of that applied to other fingers, or they may be connected. The controller may be housed on a dorsal (i.e., back) side of the glove.

According to some examples, for example as illustrated in FIG. 12, the massage device 10 may constitute part of a chair, a bed, or an apparatus configured to be secured to a chair or a bed, on which the patient sits in or lies on during the therapy. Functional elements thereof (massage assembly, pressure detection arrangement, etc.) are contained therewithin The chair, bed, or apparatus may further comprise a heating and/or a cooling arrangement. According to some examples, it may comprise a vibration arrangement.

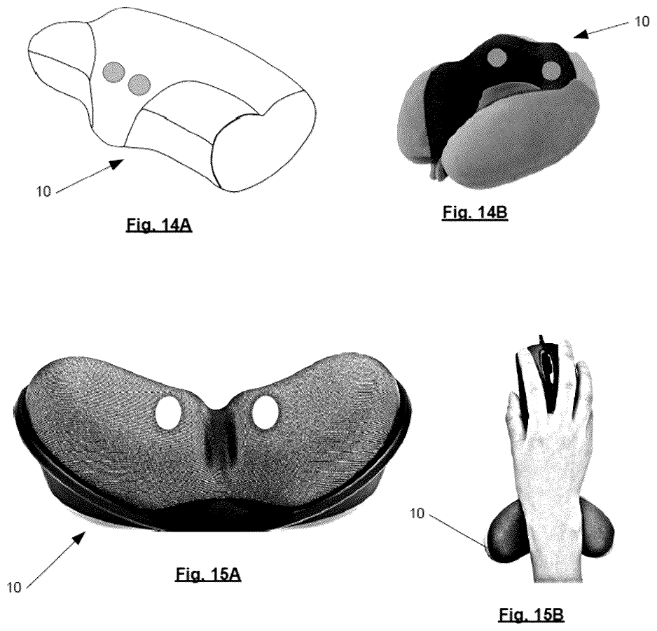

According to some examples, for example as illustrated in FIG. 13, the massage device 10 may be integrated into a shoulder compression wrap, with functional elements (massage assembly, pressure detection arrangement, etc.) contained therewithin. According to some examples, a single pressure detection arrangement is provided. According to some examples, several pressure detection arrangements are provided. The shoulder compression wrap may further comprise a heating and/or a cooling arrangement. According to some examples, it may comprise a vibration arrangement.

According to some examples, for example as illustrated in FIGS. 14A and 14B, the massage device 10 may be provided as part of a headrest and/or pillow, for example a neck pillow, with functional elements (massage assembly, pressure detection arrangement, etc.) contained therewithin. According to some examples, the massage device 10 may be configured to operate on one or both sides, e.g., comprising massage assemblies 12 facing opposite side thereof. The massage device 10 according to these examples may be useful, e.g., for treatment of headaches, suboccipital headaches, cervicogenic headaches, etc. The headrest and/or pillow may further comprise a heating and/or a cooling arrangement. According to some examples, it may comprise a vibration arrangement.

According to some examples, for example as illustrated in FIGS. 15A and 15B, the massage device 10 may be provided as part of a wrist rest, with functional elements (massage assembly, pressure detection arrangement, etc.) contained therewithin. According to some examples, the massage device 10 may be configured to operate on one or both sides, e.g., comprising massage assemblies 12 facing opposite side thereof. The massage device 10 according to these examples may be useful, e.g., for treatment of repetitive stress injuries and other similar work-related syndromes. The wrist rest may further comprise a heating and/or a cooling arrangement. According to some examples, it may comprise a vibration arrangement.

According to some examples, for example as illustrated in FIG. 16, the massage device 10 may be provided as part of a mat, with functional elements (massage assembly, pressure detection arrangement, etc.) contained therewithin. According to some examples, the mat may comprise a plurality of massage assemblies 12 at different location therewithin. The massage device 10 according to these examples may comprise illuminating elements 30 to present information to a user, e.g., related to placement of the device. The massage assemblies 12, individually and/or cooperating with other massage assemblies, may be configured to perform a deep stroking massage. The mat may further comprise a heating and/or a cooling arrangement. According to some examples, it may comprise a vibration arrangement.

According to some examples, for example as illustrated in FIG. 17, the massage device 10 may be provided as part of a ball, for example covered with radially-protruding spikes (as illustrated), with functional elements (massage assembly, pressure detection arrangement, etc.) contained therewithin. The ball may further comprise a heating and/or a cooling arrangement. According to some examples, it may comprise a vibration arrangement. According to some examples, it may further comprise a balancing arrangement, for example comprising one or more gyroscopes, configured to return the ball to a proper orientation when it deviates therefrom.

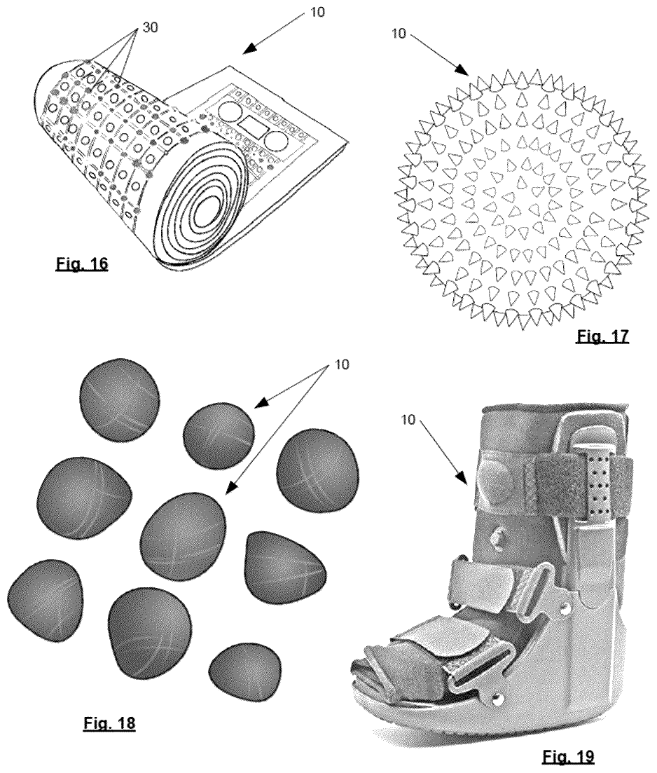

According to some examples, several examples of which are illustrated in FIG. 18, the massage device 10 may be provided as part of a weighted object, for example simulating small stones, with functional elements (massage assembly, pressure detection arrangement, etc.) contained therewithin. The weighted object may further comprise a heating and/or a cooling arrangement. According to some examples, it may comprise a vibration arrangement. The weighted objects may be configured, e.g., for placement on a patient's back to administer treatment thereto.

According to some examples, for example as illustrated in FIG. 19, the massage device 10 may be integrated into a boot, with functional elements (massage assembly, pressure detection arrangement, etc.) contained therewithin. According to some examples, a single pressure detection arrangement is provided. According to some examples, several pressure detection arrangements are provided. The massage device 10 according to these examples may be useful, e.g., for treatment of Achilles tendinopathy. The boot may further comprise a heating and/or a cooling arrangement. According to some examples, it may comprise a vibration arrangement.

It will be appreciated that the descriptions above with reference to FIGS. 9 through 19 are by way of examples only, and are not meant to be limiting. In particular, features which are described with reference to one or some of the examples are not limited to those examples; rather, they may be optionally included in any of the other examples if suitable, mutatis mutandis, without departing from the scope of the presently disclosed subject matter.

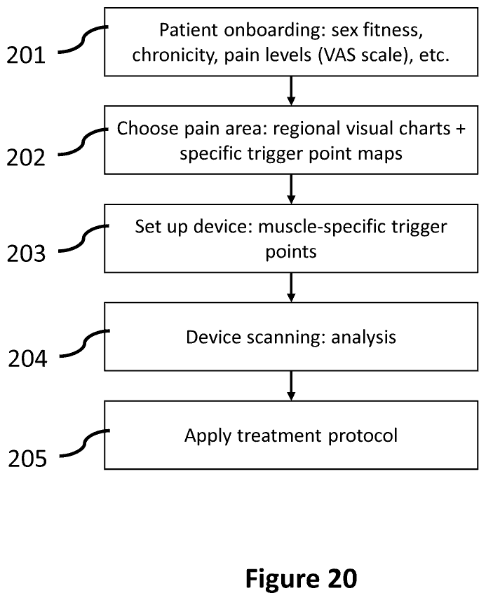

FIG. 20 shows a flowchart of methods of pain management carried out with the dynamic durometer. At first, the patients essential details are is onboard 201 onto the durometer, system, or external device. A patient is asked to select a pain area 202 via a visual chart/map or by indicating the region to a practitioner. The dynamic durometer is then directed to muscle-specific trigger points 203, or satellite trigger points associated with the pain experienced by the patient. The durometer scans the entire region, all the while measuring and collecting tissue hardness data. The device and/or the related systems perform an analysis of the data to perform various subsequent actions such as generating a pain management scheme, mapping the body and displaying it on a map, etc. Following this, a treatment protocol 205 or pain management scheme is carried out.

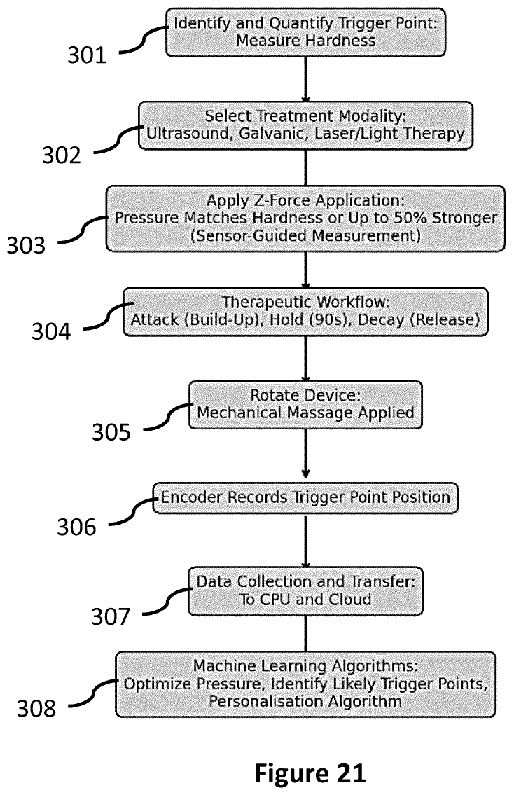

FIG. 21 shows a personalized treatment workflow. The personalized treatment workflow begins by identifying and quantifying the trigger point, measuring its hardness to assess its severity 301. Based on this assessment, the appropriate treatment modality is selected, which may include ultrasound, galvanic stimulation, or laser/light therapy 302. A controlled Z-force application is then applied, matching the measured hardness of the trigger point or exceeding it by up to (for example) 50%, with sensor-guided precision 303. The treatment follows a structured therapeutic workflow consisting of an attack (build-up) phase, a hold phase lasting 90 seconds, and a decay (release) phase 304. To further enhance the treatment, the device is rotated to apply mechanical massage to the affected area 305. An encoder records the exact position of the trigger point, allowing for future tracking and refinement 306. The collected data is then transferred to a CPU and cloud storage system for analysis 307. Machine learning algorithms process this data to optimize the applied pressure, identify potential trigger points, and refine the treatment approach through a personalized algorithm, ensuring adaptive and effective therapy 308. Artificial intelligence (AI) platforms can also be used to process and analyze data.

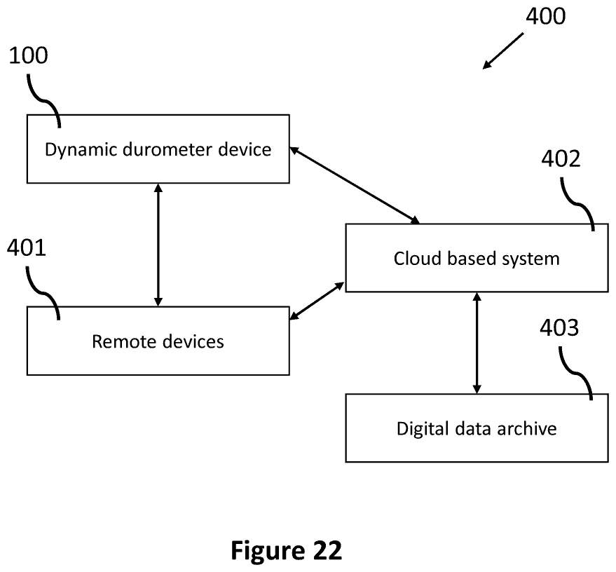

FIG. 22 shows a diagram representing the dynamic durometer system 400. Not all components are shown. The dynamic durometer system 400 comprises a dynamic durometer device 100. The dynamic durometer device 100 is connected wirelessly to external systems that include (for example) other remote devices 401 which can be synced with Cloud-based systems 402. The dynamic durometer device 100 can send and receive data to and from the remote devices 401 and the cloud based systems 402. The remote devices 401 can also communicate with the cloud based system 402. The analysis of data can occur on any platform: the dynamic durometer device 100 itself, a remote device 401, a cloud based system 402, or a combination of these. Data can be stored and recalled from a digital data archive 403 for future use. As data builds up over time, analytic programs can optimize various treatment protocols and pain management schemes through analysis tools. The analysis tools can include deep learning, AI, and machine learning.

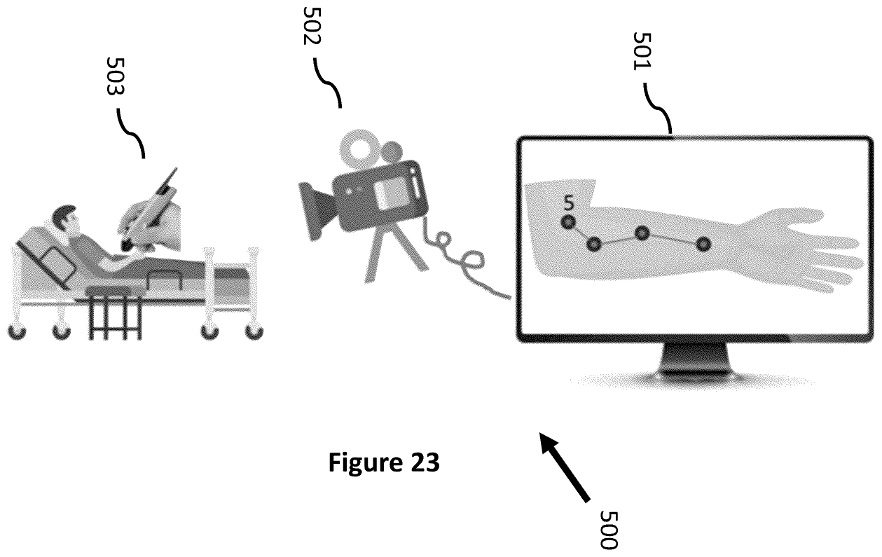

FIG. 23 shows one example of a tracking system 500. The dynamic durometer is held in a similar way as a pen and incorporates haptic feedback and wireless communication (e.g., Bluetooth). The tracking system 500 is configured to track the dynamic durometer over a human organ 503 or region such as the back or arm. A computer device application 501 is used together with image processing and a video camera 502. The computer application communicates with the dynamic durometer for hardness readings. A video processing libraries and frameworks will be used. The tracking of the dynamic durometer will be drawn 501 over the image of the body organ along with the hardness measurement. The image and data will be filed together with the patient records.

Herein the specification and appended claims, certain terms which are sometimes used in a medical context, such as “patient”, “individual”, “treatment,” etc., are used. It will be appreciated that these terms are used for convenience only to illustrate and/or define the presently disclosed subject matter, and should not be construed as making any medical or other claims regarding efficacy, nor as limiting the scope of protection to devices and/or procedures which are recognized, recommended, etc., by professional and/or governmental organizations, agencies, etc. Moreover, it will be appreciated that references herein to contact with a patient are not limited to direct contact with the skin, but include instances in which clothing, a protective cover, etc., separate the patient and the massage head without significantly affecting the pressure therebetween, mutatis mutandis.

It will be recognized that examples, embodiments, modifications, options, etc., described herein are to be construed as inclusive and non-limiting, i.e., two or more examples described separately herein are not to be construed as being mutually exclusive of one another or in any other way limiting, unless such is explicitly stated and/or is otherwise clear. Those skilled in the art to which this invention pertains will readily appreciate that numerous changes, variations, and modifications can be made without departing from the scope of the presently disclosed subject matter, mutatis mutandis.

### EXAMPLES

### Example 1

### Predefined Target Groups and Pain Management Schemes

For a predefined target group of adults (e.g., 18-90 years) with muscle tightness or post-workout soreness, the treatment with a durometer can begin with moderate pressure to assess muscle condition and relieve discomfort. The focus would be on addressing deep tissue tension and improving flexibility, especially in areas that are prone to tightness due to physical activity. As treatment progresses, the pressure can be adjusted based on pain levels and muscle response, gradually increasing to target deeper muscle layers for enhanced recovery. This dynamic approach ensures that the treatment is both effective and tailored to the individual's pain tolerance, optimizing muscle relaxation and overall mobility.

For a predefined target group of post-injury recover from a muscle strain, treatment with the dynamic durometer could begin in the acute stage with very light pressure to avoid aggravating the injury. The focus during this phase would be on soothing the affected muscle, reducing swelling, and promoting circulation to aid in healing. As the injury transitions to the subacute stage, moderate pressure can be introduced to target muscle stiffness and tension, helping to restore flexibility and range of motion. In the final recovery phase, once pain has significantly decreased, the pressure can be increased to work on deeper muscle layers, focusing on strengthening and improving muscle function. Throughout the process, the treatment would be adjusted based on the individual's pain response and progress, ensuring a gradual and safe return to full muscle strength and mobility.

### Example 2

### Utilizing Data Harvesting and Archives to Optimize Pain Management Schemes for Predefined Target Groups

Default (or baseline) pain management schemes for predefined target groups provide a structured approach to treatment, ensuring that individuals with similar characteristics receive an initial, standardized regimen that can be further optimized based on real-time feedback and specific needs. The use of big data sets, archives, and analysis tools can be used in combination to ensure optimized pain management schemes.

For example, for a particular predefined target group the dynamic durometer chip facilitates the collection of real-time data from multiple subjects, including pain levels, muscle stiffness, range of motion, and activity history, for individuals in that predefined target group. This data is stored in a cloud-based archive, where it is analyzed using optimization algorithms to identify trends and develop a baseline treatment strategy. Initially, a predetermined pressure regimen is applied to assess the affected muscle. As the treatment progresses, real-time feedback from the patient, captured by the dynamic durometer chip, enables dynamic adjustments to the pressure based on individual pain tolerance and recovery. The cloud-based system continuously updates the baseline treatment across all predefined target groups, optimizing the pain management scheme and refining it for each patient. This approach ensures that the treatment is personalized and effective, with the chip, archive, and optimization algorithms working together to enhance recovery outcomes and prevent recurrence of the condition.

Building data sets over time ensures that specific pain management schemes are tailored to specific predefined target groups.

The data analyses that arise can also subsequently be used to personalize a pain management scheme based on big data analytics. For example, although a predefined target group may require a similar baseline treatment, it could be seen that one particular age groups responds in a particular way. Thus, the pain management schemes and protocols can be modified accordingly.

### Example 3

### Predetermined Pressure Profiles for Pain Management

Predetermined pressure profiles usually comprise three stages: 1) pressure increase, 2) constant (‘sustained’) pressure, and 3) pressure decrease.

Table 1 shows a number of examples of predetermined pressure profiles with three stages, each carried out for a different period of time. In addition to these pressure profiles that are implemented by the dynamic durometer (and related systems), the dynamic durometer could carry out other functions such as vibrations.

TABLE 1
examples of predetermined pressure profiles.

| | Predetermined Pressure Profile | | |
|---|---|---|---|
| | Pressure Increase | Constant Pressure | Pressure Decrease |
| Scenario | (Shore A) | (Shore A) | (Shore A) |
| Mild Muscle Stiffness | 20 → 40 | 40 | 40 → 20 |
| | (30 seconds) | (for 1 minute) | (20 seconds) |
| Post-Workout | 20 → 60 | 60 | 60 → 30 |
| Recovery | (20 seconds) | (for 2 minutes) | (30 seconds) |
| Chronic Pain Relief | 20 → 50 | 50 | 50 → 20 |
| | (60 seconds) | (for 2 minutes) | (30 seconds) |
| Acute Muscle Injury | 20 → 30 | 30 | 30 → 20 |
| (Sprain) | (15 seconds) | (for 30 seconds) | (15 seconds) |
| Tennis Elbow | 20 → 80 | 80 | 80 → 40 |
| Treatment | (30 seconds) | (for 1 minute) | (20 seconds) |
| Post-Surgical | 20 → 40 | 40 | 40 → 20 |
| Rehabilitation | (45 seconds) | (for 1.5 minutes) | (20 seconds) |
| Deep Tissue Massage | 30 → 70 | 70 | 70 → 30 |
| | (60 seconds) | (for 1 minute) | (30 seconds) |
| Sports Injury | 20 → 50 | 50 | 50 → 20 |
| (Muscle Spasm) | (5 seconds) | (for 45 seconds) | (20 seconds) |

As data is collected over time, these predetermined pressure profiles can change, in particular for different predetermined target groups. Furthermore, additional stages can be added or removed to the pain management scheme.

Although Table 1 shows individual pressure profiles, these pressure profiles can be used as part of a longer-term series of treatment sessions which can vary over time depending on the individual's response to treatment and other factors.

Furthermore, for example, if an individual requires a more intensive treatment, the pressure increase can be greater or considered more ‘aggressive’, as shown in Table 2.

TABLE 2
differences in pressure profile for different muscle stiffness levels.

| | Predetermined Pressure Profile | | |
|---|---|---|---|
| | Pressure Increase | Constant Pressure | Pressure Decrease |
| Scenario | (Shore A) | (Shore A) | (Shore A) |
| Mild Muscle | 20 → 40 | 40 | 40 → 20 |
| Stiffness | (30 seconds) | (for 1 minute) | (20 seconds) |
| Severe Muscle | 20 → 80 | 80 | 80 → 20 |
| Stiffness | (20 seconds) | (for 1 minute) | (20 seconds) |

In one embodiment, the term “a” or “one” or “an” refers to at least one. In one embodiment the phrase “two or more” may be of any denomination, which will suit a particular purpose. In one embodiment, “about” or “approximately” may comprise a deviance from the indicated term of +1%, or in some embodiments, −1%, or in some embodiments, ±2.5%, or in some embodiments, ±5%, or in some embodiments, ±7.5%, or in some embodiments, ±10%, or in some embodiments, ±15%, or in some embodiments, ±20%, or in some embodiments, ±25%.

Those skilled in the art to which this invention pertains will readily appreciate that numerous changes, variations, and modifications can be made without departing from the scope of the presently disclosed subject matter, mutatis mutandis.

1. A dynamic durometer device for pain management in an individual, the device comprising:
an indentor assembly comprising at least one displacement sensor, and
configured to measure the indentation depth when pressed on the skin of the individual;
a roller assembly head attached at the distal end of the indentor assembly and configured to maintain contact with the skin whilst the indentor assembly measures the indentation depth; and
a chip comprising a controller configured to calculate tissue hardness based on the indentation depth.

2. The dynamic durometer of claim 1 wherein the controller is further configured to:
calibrate the dynamic durometer device after the indentor assembly selectively applies pressure to the skin of the individual to provide a baseline tissue hardness value;
record tissue hardness values at different locations of the individual; and
identify regions of elevated tissue hardness values in comparison with the baseline tissue hardness value.

3. The dynamic durometer of claim 1 wherein the roller assembly head comprises at least one rollerball head, at least one axle-mounted cylindrical roller, or a combination thereof.

4. The dynamic durometer of claim 3 wherein the at least one displacement sensor is operatively coupled to each of the at least one rollerball head, the at least one axle-mounted cylindrical roller, or a combination thereof.

5. The dynamic durometer of claim 4 further comprising a triangulation system comprising at least three displacement sensors and the controller, wherein the triangulation system is configured to identify regions of elevated tissue hardness values with greater resolution, enhance the accuracy and precision of the indentation depth measurement, or a combination thereof.

6. The dynamic durometer of claim 1 wherein the roller assembly head is coupled to a gimbal system or a ball-and-socket system, and thereby configured to rotate about multiple axes to enable multidirectional movement.

7. The dynamic durometer of claim 1 further comprising a display unit configured to display the depth of indentation, tissue hardness value, or a combination thereof.

8. The dynamic durometer of claim 2 wherein the at least one rollerball head has a diameter ranging between 0.5 cm to 5 cm.

9. The dynamic durometer of claim 1 further comprising a housing structure comprising the indentor assembly within it and wherein the roller assembly head protrudes from the housing structure.

10. The dynamic durometer of claim 1 further comprising a handle attached to the housing structure.

11. The dynamic durometer of claim 1 further comprising an ultrasound unit operatively coupled to the roller assembly head and configured to deliver ultrasound, vibrations, or a combination thereof, to the individual when the roller assembly head is in contact with the individual.

12. The dynamic durometer of claim 1 wherein the roller assembly head further comprises a light source configured to deliver light into the skin of the individual when the roller assembly head is in contact with the individual.

13. The dynamic durometer of claim 12 wherein the light source is a laser.

14. The dynamic durometer of claim 1 further comprising a temperature regulation system operatively coupled to the roller assembly head and configured to maintain a predetermined temperature for the roller assembly head.

15. The dynamic durometer of claim 1 further comprising a gyrometer configured to monitor the orientation, spatial rotation, spatial location, and angular velocity of the dynamic durometer.

16. The dynamic durometer of claim 1 further comprising an indicator element operatively coupled to the controller and configured to provide haptic, visual, or auditory feedback to a user upon detection of a predetermined tissue hardness.

17. The dynamic durometer of claim 1 wherein the controller is further configured to direct the indentor assembly to selectively apply a predetermined pressure at or near one or more locations with elevated tissue hardness, thereby reducing pain in the individual.

18. The dynamic durometer of claim 1 further comprising a power source.

19. The dynamic durometer of claim 1 wherein the chip further comprises:
an analog-to digital converter (ADC) configured to convert tissue hardness data into digital data;
an encoder configured to encode the digital data, resulting in encoded digital data;
a memory unit configured to store the encoded digital data; and
a communication module comprising a wireless transmitter operatively coupled to the controller and configured to send and receive the encoded digital data to an external system;
or a combination thereof.

20. A pain management system comprising:
the dynamic durometer of claim 19; and
an external system configured to send and receive the encoded digital data.
